(19) United States
(12) Patent Application Publication
(10) Pub. No.: US 2013/0142310 A1
(43) Pub. Date: Jun. 6, 2013

(54) DYNAMIC MULTI-AXES TRAJECTORY OPTIMIZATION AND DELIVERY METHOD FOR RADIATION TREATMENT

(71) Applicants: Benjamin Pooya Fahimian, Menlo Park, CA (US); Lei Xing, Palo Alto, CA (US); Sarah Geneser, Menlo Park, CA (US); Dimitre Hristov, Stanford, CA (US)

(72) Inventors: Benjamin Pooya Fahimian, Menlo Park, CA (US); Lei Xing, Palo Alto, CA (US); Sarah Geneser, Menlo Park, CA (US); Dimitre Hristov, Stanford, CA (US)

(73) Assignee: The Board of Trustees of the Leland Stanford Junior University, Palo Alto, CA (US)

(21) Appl. No.: 13/490,450

(22) Filed: Jun. 6, 2012

Related U.S. Application Data
(60) Provisional application No. 61/493,977, filed on Jun. 6, 2011.

(51) Int. Cl. A61N 5/10

(52) U.S. Cl. CPC A61N 5/103
USPC 378/65

(57) ABSTRACT

Embodiments of the present invention provide methods for improved radiation treatment and imaging of solid cancers utilizing radiation beam trajectory optimization techniques to obtain conformal radiation coverage of tissue that is targeted to receive radiation, while minimizing exposure of healthy tissue and organs to harmful, unnecessary radiation.

### 1. RELATED APPLICATION

This application claims priority and other benefits from U.S. Provisional Patent Application Ser. No. 61/493,977, filed Jun. 6, 2011, entitled “Method of partial breast irradiation and imaging”. Its entire content is specifically incorporated herein by reference.

### 2. TECHNICAL FIELD OF THE INVENTION

The present invention relates generally to external-beam radiation treatment of solid cancers, and more specifically, to the use of radiation beam trajectory optimization techniques to obtain conformal radiation coverage of tissue that is targeted to receive radiation, while sparing normal tissue.

### 3. BACKGROUND

External-beam radiation treatment relies on the use of externally administered high-energy ionizing particles such as x-rays, gamma rays, neutrons and protons to target and destroy the genetic information in cancerous cells. Since cancerous cells are fast-growing and possess a reduced ability to repair errors in their genetic information, radiation therapy is a treatment modality that is effective and utilized in treating various solid cancers such as breast cancer, pancreas cancer or liver cancer, just to name a few.

To spare neighboring, healthy tissues, radiation beams are aimed from several angles of exposure to intersect at the cancerous site in order to achieve a particularly high exposure of radiation in the cancerous tissues. The inclusion of additional degrees of freedom in linear accelerator delivery hardware has continuously improved the efficacy of radiation treatments, as evidenced by the progression from conformal therapy to intensity modulated radiation therapy via the development of multi-leaf collimators as well as the progression from intensity modulated radiation therapy to volumetric modulated arc therapy by continuously driving the gantry during treatment. Recent advances in clinical linear accelerator control make it possible to drive the various tools used in the course of external beam-radiation treatment, such as radiation couch and gantry, in concert to produce complex three-dimensional radiation delivery trajectories.

One of the most challenging topics facing the prospect of radiation treatment is the development of late tissue toxicities resulting from non-conformal irradiation of large volumes of normal, i.e. noncancerous tissue, such as normal breast tissue, due to the poor radiation control in the course of conventional radiation treatment.

It would be highly desirable to have improved methods available to produce conformal radiation dose distributions so that the direct radiation dose to cancer is increased, while the normal tissue radiation dose is reduced, enhancing the therapeutic ratio of radiation treatment in solid cancer treatment. The present application addresses this issue.

### 4. SUMMARY

One aspect of the invention provides a method for radiation treatment of solid cancers by optimizing radiation beam trajectories and creating non-colliding dynamic trajectories to produce conformal radiation dose distributions in the cancerous and to be treated tissue. These optimization methods are based on a coordinated movement of various tools used in the course of external beam-radiation treatment, such as radiation couch and gantry, to produce complex three-dimensional radiation delivery trajectories.

In one embodiment, this method is applied to the treatment of breast cancer in the context of accelerated partial breast irradiation: by creating complex coronal arcs around the ipsilateral breast of a recipient of partial breast irradiation in prone position a conformal radiation dose distribution in the diseased breast tissue is achieved.

Another aspect of the invention provides a treatment planning approach for radiation treatment of solid cancers for producing and optimizing the trajectory of multiple treatment axes and achieving highly conformal radiation dose distribution utilizing i) coordinated movements of various tools used in the course of external beam-radiation treatment including dynamic rotation, translation, and tilt of the couch, rotation of the gantry and collimator, and synchronized modulation of the source collimation and ii) optimization algorithms to determine non-colliding gantry and couch trajectories.

Another aspect of the invention is an improved method for total body irradiation (TBI) combining motion of the couch and inversed planned intensity modulated beam delivery to minimize the exposure of healthy organs to harmful and unnecessary irradiation in the course of TBI.

Another aspect of the invention is an improved method for total lymphoid irradiation (TLI) combining motion of the couch and inversed planned intensity modulated beam delivery to minimize the exposure of healthy organs to harmful and unnecessary irradiation in the course of TLI.

Another aspect of the invention is a method for producing complex non-coplanar curving volumetrically modulated arcs through the use of concurrent couch and gantry motion to minimize the exposure of healthy organs to harmful and unnecessary irradiation.

Another aspect of the invention is an imaging method in which, using rotation and translation of the couch and on-board imagers, image projections are taken of the subject at different relative angles. The projections at different relative angles generated through, for example, rotation of the couch and gantry are then reconstructed using tomography or tomosynthesis techniques to provide imaging assessment of a region of interest in the subject.

The above summary is not intended to include all features and aspects of the present invention nor does it imply that the invention must include all features and aspects discussed in this summary

### 5. INCORPORATION BY REFERENCE

All publications mentioned in this specification are herein incorporated by reference to the same extent as if each individual publication or patent application was specifically and individually indicated to be incorporated by reference.

### 6. BRIEF DESCRIPTION OF THE DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 represents, in accordance to an embodiment of the present invention, a graphical representation of the conventional linear accelerator (LINAC) geometry for radiation treatment, and the dynamic modes of motion across the multiple axes utilized in the methods of the present invention. Specifically, in the herein described approach to radiation treatment the (radiation) couch can dynamically move, as shown, during modulated beam delivery through rotations, translations, and tilts in a coordinated manner with motion of the (rotation) gantry and beam modulation through dynamic collimation. 100 denotes the radiation gantry, 101 the radiation treatment couch (showing rotation, translation, and tilts), and 102 denotes the synchronized modulated collimation, e.g. multi-leaf collimator or MLC.

FIG. 2 represents the motion described in FIG. 1, as viewed from the top of the room looking down. 100 denotes the radiation gantry, 101 the radiation treatment couch (showing rotation, translation, and tilts), and 102 denotes synchronized modulated collimation, e.g. multi-leaf collimator or MLC. The arrows indicate examples of dynamic motion of the axes during synchronized modulated beam delivery.

FIG. 3 demonstrates plots of complex trajectories generated from embodiments of the present invention; the trajectories were derived from the optimization algorithm outlined in Table 1. The top panel represents a 4-dimensional Computer Aided Design (CAD) visualization environment utilized in the treatment trajectory optimization enabling analysis of the treatment trajectory in conjunction of the beams eye view generated with the patient model (CT scan) and target volume, as delineated in Example 1. The middle panel represents plots of the couch trajectories, as viewed from the top of the room for derived motion from said methods of trajectory optimization for the prone partial breast irradiation embodiment (see Example 1). The bottom panel shows photos of the reduction to experiment at different times during the trajectory as indicated.

FIG. 4 illustrates, in accordance to an embodiment of the present invention, the setup and radiation treatment plan results for the prone partial breast irradiation technique, as described in Example 1. (a) Patient setup during simulation and delivery on a breast board. (b) Illustrates the beam geometry relative to target (breast lumpectomy cavity) for the optimized treatment trajectory depicted in FIG. 3, showing the wide-angular irradiation that can be achieved through the described methods, while completely eliminating irradiation through the thorax and contralateral breast tissue. (c) and (d) show dose distribution of the conventional 5 field IMRT plan, and the proposed method respectively, visualized at the clinically relevant 50% threshold, showing the proposed technique results in significant reduction of collateral dose to the normal tissue. (e) shows a comparison of the corresponding normal breast tissue dose volume histograms (DVHs), and reduction of dose parameters associated with toxicity through the proposed method.

FIG. 5 shows an exemplary implementation and delivery of a dynamic prone partial breast irradiation plan, as described in FIG. 4(d) onto film and ion chamber and as detailed in Example 1. The accuracy of the method was quantified relative to the planned expected dose from the treatment planning software using (a) Gamma pass/fail criteria of 3%/3 mm, (b) comparison of planned and delivered isodoses, and comparison of the vertical (c) and horizontal (d) profiles of the delivered and planned dose distribution.

FIG. 6(a) illustrates an exemplary dynamic collimation during beam delivery using a Multi-Leaf Collimator (MLC) to track and irradiate the target. FIG. 6(b) shows variations of the partial breast irradiation technique in which the gantry moves dynamically in addition to the couch rotation and translation (in contrast to the implementation in FIGS. 3 and 4 which the gantry is fixed at two angles and the MLCs are purely used for tracking).

FIG. 7 illustrates another preferred embodiment of the present invention, in which the methods of the present invention are utilized to produce dynamic trajectory modulation, using the combined motion of the couch and gantry, in order to create complex non-coplanar curving arc-like irradiation paths that irradiate a solid cancer, while minimizing the exposure of healthy organs to harmful and unnecessary irradiation.

FIG. 8 illustrates another preferred embodiment of the invention for irradiating extended anatomical portions of the body, as in common types of radiation treatments such as Total Body Irradiation (TBI) that is used for immune system suppression prior to bone marrow transplantation, or Total Lymphoid Irradiation (TLI). Specifically, a sagittal volumetrically modulated arc is delivered in conjunction with translation of couch to allow the radiation to cover the total body or extended anatomical region in a single partial arc delivered from the anterior (as depicted) or alternatively or additionally from the posterior. Inverse planning and intensity modulated delivery are uniquely utilized for robust optimization of the dose.

### 7. BRIEF DESCRIPTION OF THE TABLES

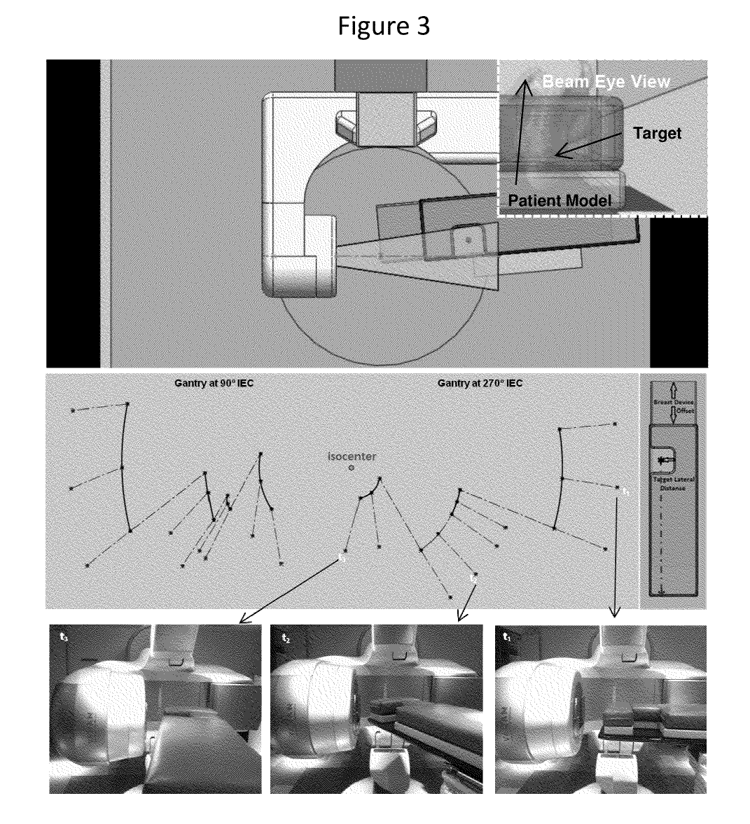

Table 1 outlines the pseudo-code for the optimization algorithm used to generate the prone partial breast irradiation trajectory, as described in FIGS. 3-5.

TABLE 1
Trajectory Optimization Algorithm Pseudo-Code for
the Prone Partial Breast Irradiation Embodiment

ALGORITHM 1{

| | |
|---|---|
| SET | Gantry ROT = 90°; Couch ROT =270°; Couch LAT= limit; |
| | Couch LONG= limit; |
| | Couch VERT such that P_{MLC} ∩ P_{PTV} ≠ Ø ; |

WRITE // Record initial delivery control point
Tₜ(C): ³ → ³ // Increment couch to new position
SUB-ALGORITHM 1{

IF C ∩ L = Ø // No collision between couch or LINAC,

// But may not be useful if target outside BEV or MLC
limits

IF P_{MLC} ∩ P_{PTV} ≠ Ø //Target is in beam's eye view
WRITE //Record as delivery control point;
END

END}

| | |
|---|---|
| Tₜ(C): ³ → ³; | // Increment couch either because control point found, |
| | // collision detected, or target outside BEV |

WHILE (Couch LONG and ROT < Limit) ; GOTO SUB-ALGORITHM 1
REPEAT for Gantry ROT=270°
}

### 8. DETAILED DESCRIPTION

Before describing specific embodiments of the invention, it will be useful to set forth definitions that are utilized in describing the present invention.

### 8.1 Definitions

The practice of the present invention may employ conventional techniques of radiological and radiation treatment, which are within the capabilities of a person of ordinary skill in the art. Such techniques are fully explained in the literature. For definitions, terms of art and standard methods known in the art, see, for example, Faiz M. Khan, “The Physics of Radiation Therapy”, Fourth Edition, Lippincott Williams & Wilkins, Baltimore and Philadelphia, 2009, and Jerold T. Bushberg et al. “The Essential Physics of Medical Imaging”, Second Edition, Lippincott Williams & Wilkins, Baltimore and Philadelphia, 2001. Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a” and “the” include plural referents, unless the context clearly dictates otherwise

The terms “irradiation” and “radiation treatment” are used interchangeably herein and relate to the exposure of a subject suffering from a solid cancer to radiation for the purpose of destroying cancerous cells of the targeted tissue.

The terms “radiation gantry” and “gantry” are used interchangeably herein and relate to a movable device containing the radiation producing source for radiation delivery.

The terms “radiation couch” and “couch” are used interchangeably herein and relate to a device for supporting the recipient of a radiation treatment during the treatment.

The term “subject”, as used herein, refers to a member of a species of mammalian origin.

The terms “accelerated partial breast irradiation”, “partial breast irradiation”, “APBI” and “PBI” are used interchangeably herein and relate to a type of radiation therapy that a) is applied only to the diseased part of the breast and b) uses a higher dose over a shorter time than conventional whole-breast radiation therapy does.

“Conformal radiation coverage of tissue”, as used herein, means radiation coverage that is shaped to the contours of the tissue that is to be treated with radiation.

The term “conformal dose”, as used herein, is the radiation dose that is used in the context of conformal radiation therapy. Conformal radiation therapy utilizes three-dimensional images of a malignant neoplasm in order to target the highest possible radiation dose at the neoplasm, while minimizing exposure of surrounding healthy tissue.

The term “linear accelerator” or “LINAC” relates to a device that generates energy for external beam radiation cancer treatment. External beam radiation is the most common form of radiotherapy, where the recipient of radiotherapy is positioned on a conventionally stationary couch and an external source of radiation is targeted towards a selected part of that recipient's body.

The term “couch trajectory”, as used herein, relates to the path of the moving couch (radiation couch) through space during dynamic trajectory radiation therapy which includes rotation, translation, and tilt of the couch.

The term “gantry angle”, as used herein, defines the respective angle to which the gantry is rotated.

The term “gantry speed”, as used herein, defines the speed at which the gantry is rotated.

The term “multi-leaf collimator”, as used herein, relates to a device that is used on linear accelerators to produce conformally shaped radiation beams. It consists of individual leaves of a high atomic numbered material and moves in and out of the path of a radiation beam in order to block it.

The term “multi-leaf collimator rotation”, as used herein, relates to the relative position of each individual leaf of a multi-leaf collimator at a range of various gantry angles.

The term “monitor unit”, as used herein, defines a measure of machine output of a linear accelerator in radiation therapy.

### 8.2 Radiation Treatment

Radiation treatment is an important treatment modality in the curative and palliative management of subjects who suffer from a solid cancer such as cancer of the breast, prostate, liver and the like. Treatment planning involving careful determination of tumor volume and the borders of surrounding healthy breast tissue are critical for delivering focused beams of radiation to diseased tissues, while minimizing radiation effects on surrounding health tissues. Linear accelerators are commonly utilized to produce external beam radiation which is high-energy radiation consisting of focused, fast-moving subatomic particles treatment. The availability of multi-leaf collimators (MLC) on modern linear accelerators has made possible the delivery of multiple and complex portal geometries necessary for intensity modulated radiation therapy.

### 8.2.1 Radiation Therapy Approaches That Integrate Three-dimensional Imaging Technology for Visualizing the Diseased Tissue to Increase Efficiency and Precision of Radiation Therapy.

Intensity-modulated radiation therapy (IMRT). Intensity-modulated radiation therapy utilizes computer-generated images to visualize the size and shape of a malignant neoplasm and, subsequently aim beams of radiation of differing intensities at the neoplasm from various angles. Volumetric modulated arc therapy (VMAT). Volumetric modulated arc therapy represents delivery of intensity modulated beams during an arc rotation of the source about a subject on a stationary couch.

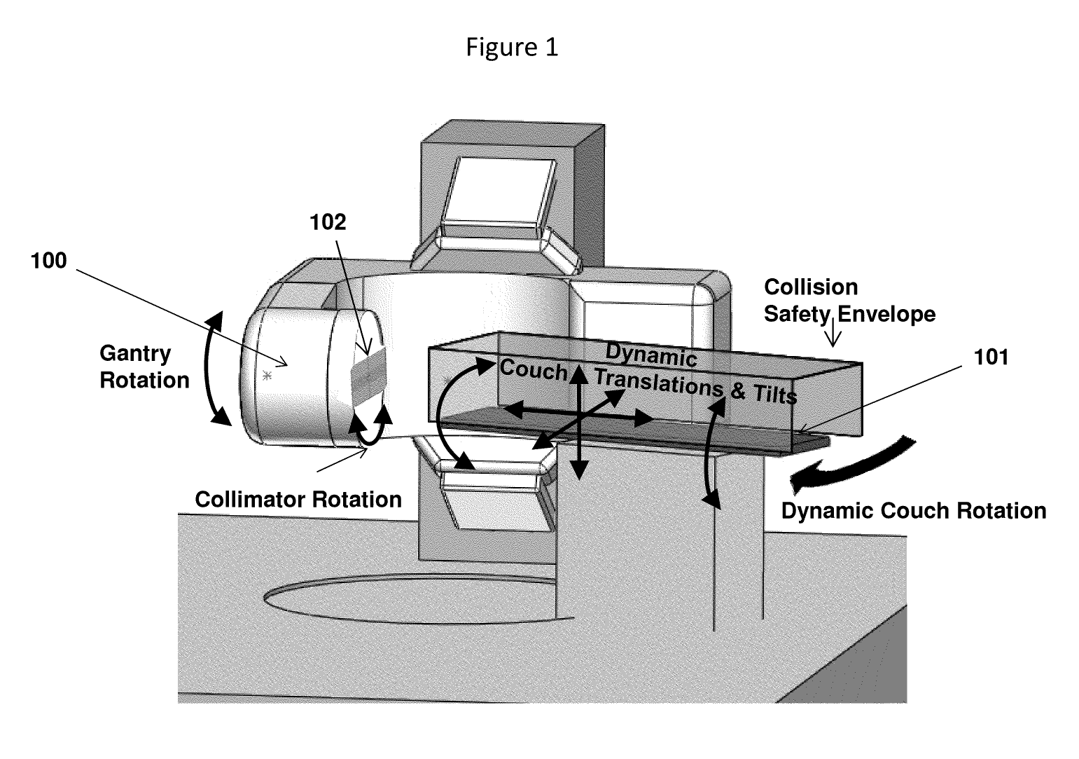

Dynamic trajectory radiation therapy (DTRT), as described herein, is a method that fully leverages the additional delivery control and degrees of freedom that couch motion offers. DTRT can be used to define couch motion during delivery in a manner that spares critical organs by providing beam angles that avoid intersect critical tissues, and provide more conformal radiation through wide-angular irradiation of beam directions otherwise not possible, while still ensuring delivery of a conformal dose to the tumor target. FIG. 1 illustrates degrees of freedom and types of dynamic motions during beam delivery that are utilized in DTRT. In order to achieve successful DTRT treatment plans, methods have been developed to determine the optimal (1) couch position, (2) gantry angle, (3) collimator rotation, (4) MLC apertures, and (5) monitor units necessary for successful DTRT treatments.

Intensity modulated radiation therapy (IMRT) relies on non-uniform radiation exposure to target the highest radiation exposure to the diseased tissues, for example, the post-lumpectomy cavity. Multi-leaf collimators, which consist of narrow leaves and which can form custom-shaped portals, can move in and out of the beam portal under precise computer guidance during beam-on time and can so generate a desired non-uniform intensity pattern that produces a uniform dose to the target. The leaves of the multi-leaf collimator might also slide during radiation exposure which adjusts the intensity of portions of the beam.

IMRT can be employed with either multiple fixed gantry (linear accelerator treatment head) positions or a rotation gantry. With multiple fixed gantry positions, optimal multiple beam angle and optimal table configurations are chosen. With a rotation gantry technique, the gantry swings around the subject in an arc configuration while during beam-on time, and the field shape and intensity are continually modified.

By targeting radiation to only a limited volume of breast tissue adjacent to the lumpectomy cavity, it may be possible to give a larger dose per fraction in a shorter time period, while still ensuring tumor control and favorable cosmetic results.

### 8.3 Treatment Planning for Dynamic Trajectory Radiation Therapy (DTRT)

A DTRT plan comprises a set of control points, (G(MU),T(MU),C(MU),MLC(MU)), that prescribe the gantry angle, couch position, collimator rotation, and MLC leaf positions in terms of the cumulative delivered MU. Treatment planning for DTRT proceeds in two main steps (i) determining the treatment trajectory, and (ii) the multi-leaf collimator (MLC) leaf positions and monitor units (MU) that produce optimal delivered dose characteristics.

First, the treatment trajectory must be defined. To do this, the gantry angle, Gᵢ, couch position, Tᵢ, and collimator rotation, Cᵢ, are defined for each i^{th} position along the treatment path. The delivery trajectory, (Gᵢ,Tᵢ,Cᵢ) for i=1 . . . N, can be chosen to avoid delivery angles that pass through especially sensitive organs, spread the dose over greater number of total angles, etc. Couch positions and gantry angles must be chosen so that throughout the treatment (a) the planned target volume is within the multi-leaf collimator (MLC) aperture field of view and (b) no collisions occur between the gantry and either the subject or the couch. Note that the choice of collimator rotation can affect (a) and should be chosen appropriately. Moreover, because the couch and gantry can be moved independently of one another, the space of possible couch and gantry position combinations is quite large. It is therefore necessary to restrict this space when considering potential couch and gantry trajectories during DTRT treatment. Because of the large the effort associated with developing optimal trajectories, we propose that therapy trajectory be chosen for specific treatment sites and disease of interest as opposed to subject-specific trajectories.

Predetermining the gantry speed during delivery significantly simplifies optimization at the cost of reducing the degrees of freedom and possibly restricting plan quality. In the case of breast API, which only uses two gantry angles throughout the entire treatment, and DTRT plans relying on IMRT, the gantry is stationary during the treatment and is therefore predetermined. Despite this apparent restriction, excellent plans are achieved. It is also possible to design a plan in which the trajectories are optimized as part of the dose delivery optimization step. In this case, it is important to define a set of acceptable gantry, couch, and collimator trajectories as a pre-computing step. However, simultaneously optimizing for both the dynamic trajectory and dose significantly complicates optimization.

Second, once the treatment delivery dynamics are determined, the MLC beam apertures, MLCᵢ, and delivered monitor units, MUᵢ, must be optimized to achieve PTV dose prescription, maximize dose conformality, and minimize the dose to critical organs. To be consistent with Varian's TrueBeam control points, the resulting optimized treatment plan comprising of delivery dynamics for each of the N positions along the delivery trajectory, (Gᵢ,Tᵢ,Cᵢ,MLCᵢ,MUᵢ), can be expressed as a function of MU: (G(MU),T(MU),C(MU),MLC(MU)).

### 8.3.1. Collision Detection and Trajectory Optimization

The couch geometry and initial position are defined as a set of points, C₀∈³, in three-dimensional space. Note that C₀ includes the couch geometry, subject volume, immobilization device, and target geometry, all of which are encompassed in a safety envelope as shown in FIG. 1a. The couch position at any given time, t, is modeled as a rigid transformation acting on the initial couch position, C(t)=T(C₀,t): (³,)→³, that acts on the couch geometry and allows translation in the x-, y-, and z-axes and rotation in the x-y plane only. The LINAC geometry and positions defined as a set of points, L(t)∈³, and depends on the specific time-dependent orientation of the LINAC. Subsequently, under a non-collisional couch and LINAC state, the intersection of the two closed sets is the null set, i.e., C̅∩ L̅=Ø, while a collision is detected if C̅∩ L̅≈Ø. Besides a non-collisional state between the couch and LINAC components, the target must be in the field of view of the MLCs whenever the beam is on during delivery. A projectional operator is defined as P: ³→², that projects the outline of the MLCs and the PTV at the specific couch angle to the sagittal plane at isocenter, denoted as P_{MLC}∈² and P_{PTV}∈², respectively. For a valid beam delivery control point, it is required that P_{MLC}∩P_{PTV}≈Ø.

This method verifies that for each plan trajectory control point, there are no collisions. It does not, however, check for collisions during the motion from one control point to the next. To ensure such collisions will not occur, it is necessary to create a model of the linear accelerator capable of detecting collisions and target position with respect to the collimator field of view given the linear accelerator and safety envelope geometry, couch and target position, gantry angle, and collimator rotation.

To design a dynamic delivery trajectory, starting and stopping control points are set to spread the dose and avoid critical structures. Control points are then interpolated between the starting and stopping points. Each control point is then checked for conflicts (e.g. collisions or the target moving outside the collimator field of view). If either conflict occurs, an additional control points can be inserted into the path that move the couch or gantry to remove the conflict, and the remaining path is processed recursively in a similar manner. An exemplary pseudocode for generating dynamic trajectories for partial breast arc irradiation is provided in Table 1, supra.

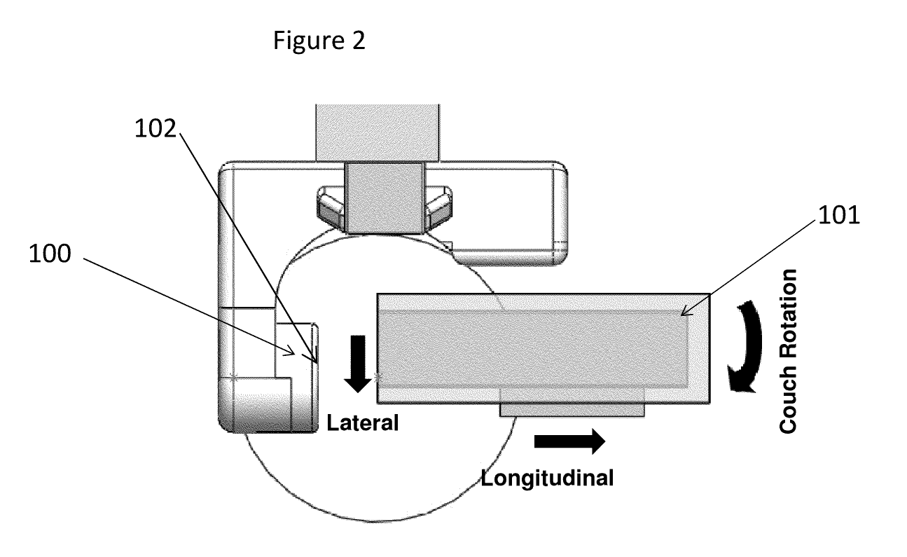

In one embodiment trajectory, optimization is performed and aided graphically by the user through the use of 4-dimensional Computer Aided Design (CAD) modeling, which is the basis for the figures generated in FIGS. 1, 2 and in the top panel of FIG. 3. Using such a model, the trajectory, beams eye view, collisions, and dynamics of the entire system can be visualized, analyzed, and optimized FIG. 3 shows an exemplary embodiment for prone partial breast irradiation (see top panel of FIG. 3) which constitutes a screen capture of the moving 4D model; the patient CT scan and the target can be imported into the trajectory optimization visualization environment and the beams eye view was dynamically analyzed as a function of time as shown.

In other embodiments, the trajectory can be designed by the user by any means, and then sub-sampled and run through the below methods of the plan optimization based on the desired user defined trajectory.

### 8.3.2. Generalized DTRT Plan Optimization

The total delivered dose can be calculated as ΣᵢMUᵢ×D(Gᵢ,Tᵢ,Cᵢ,MLCᵢ), where D(Gᵢ,Tᵢ,Cᵢ,MLCᵢ) is a function that computes the dose for the gantry angle, couch position, collimator angle, and MLC beam aperture or beamlet field at the i^{th} time point along the treatment trajectory. When the precise delivery path is determined prior to dose optimization, the couch position at each gantry angle can be incorporated into the dose calculation. This is highly beneficial in that treatment planning can then proceed as it does for conventional IMRT or VMAT, so long as the dose dynamics are computed for the appropriate gantry angle, couch position, and collimator rotation, for each control point, (Gᵢ,Tᵢ,Cᵢ), along the delivery path.

### 8.3.3 DTRT Arc Therapy Plan Optimization

Here a mathematical framework is presented for incorporating dynamic couch position, rotation, and tilt into treatment planning optimization for dynamic arc therapy. Given a delivery path consisting of N control points describing the gantry angle, couch position, and collimator rotation for delivery, it remains to optimize the monitor units and MLC apertures, (MUᵢ,MLCᵢ), for each of the control points. To improve optimization efficiency, the control points are first sub-sampled and the MLC apertures are initialized to the intersection of the target volume with an additional small margin, projected onto the collimator of the linear accelerator.

The monitor units are then optimized over the entire sub-set of control points to produce the ideal dose distribution for the initial apertures as follows:

$$minimize \{ {f}_{1} ( d ) , {f}_{2} ( d ) , … , {f}_{M} ( d ) \} \\ ( MU , MLC ) \\ subject to d = {\sum }_{i = 1}^{N} {MU}_{i} ( {D}_{i}^{*} {MLC}_{i} ) \\ d ∈ A \\ {MLC}_{i} \geq 0 ∀ i = 1 , … , N \qquad ( 1 )$$

where fᵢ are the M dose objective functions for the structures of interest, A is the convex set of dose constraints, and Dᵢ is the dose matrix for the i^{th} control point with gantry angle, Gᵢ, collimator rotation, Cᵢ, and couch position, Tᵢ. Dᵢ acts on the MLC aperture, MLCᵢ, to produce the dose distribution, dᵢ, at the i^{th} control point. The total dose consists of sum of the doses, dᵢ, at the N control points weighted by MUᵢ, the monitor units delivered at the i^{th} control point. Equation 1 is solved several times, first with a sub-sampling of N control points and then at each subsequent solution iteration, it is solved at a greater number of control points, until it is solved over all N control points. For the initial solution iteration, the apertures are initialized to the target volume plus an additional small margin projected onto the MLC. The MUs and MLC apertures are then optimized using an iterative optimization method (e.g. simulated annealing) for the subset of control points, with checks performed at each potential solution step to ensure that the control points comply with the machine constraints (e.g. maximum leaf speed, maximum dose rate, etc.). When progressing from the j^{th} level of sub-sampling to the (j+1)^{th} level, the end solution of the j^{th} level is interpolated to the (j+1)^{th} level and serves as an initialization for that level. The optimization proceeds until Equation 1 is solved for all N control points.

Equation 1 is solved several times, first with a sub-sampling of control points; at each subsequent solution iteration, it is solved at a greater number of control points, until it is solved over all control points. For the initial solution iteration, the apertures are initialized to the target volume plus an additional small margin projected onto the MLC. The MUs and MLC apertures are then optimized using an iterative optimization method (e.g. simulated annealing) for the subset of control points, with checks performed at each potential solution step to ensure that the control points comply with the machine constraints (e.g. maximum leaf speed, maximum dose rate, etc.).

When progressing from the j^{th} level of sub-sampling to the (j+1)^{th} level, the end solution of the j^{th} level is interpolated to the (j+1)^{th} level and serves as an initialization for that level. The optimization proceeds until Equation 1 is solved for all N control points.

Though in the description above, the error metric for dose optimization is formulated to meet dose volume constraints for the structures of interest, it can also be formulated to penalize the mean squared difference between the prescribed and computed dose.

### 8.3.4 DTRT IMRT Plan Optimization

Here the mathematical framework for incorporating dynamic couch motion into IMRT treatment planning optimization is formulated. Given a dynamic trajectory IMRT delivery plan consisting of N delivery angles, the delivery path consists of N control points describing the gantry angle, couch position, and collimator rotation for delivery. The dynamic trajectory IMRT plan is obtained by optimizing the MLC fluence map for each of the control points as follows:

$${minimize}_{w} {‖ d - {D}_{Rx} ‖}_{2}^{2} \\ subject to d = {\sum }_{i} {D}_{i} {w}_{i} \\ 0 \leq w \leq {w}_{ma x} \\ {D}_{m i n} \leq d \leq {D}_{ma x} \qquad ( 2 )$$

where Dᵢ is the dose fluence matrix that computes the dose resulting from wᵢ for the specific gantry angle, couch position, and collimator rotation for the i^{th} control point. The vector, w, is the concatenation of the beamlet intensities that form the N fluence maps, wᵢ, at the i^{th} control point. D_{Rx}, Dₘᵢₙ, and Dₘₐₓ are the prescribed, minimum, and maximum doses for the structures of interest. Equation 2 is optimized using convex methods and the resulting N beamlet fluence maps are processed using leaf sequencing to determine MLC apertures that achieve the optimized fluence maps. While in the description above, the error metric for dose optimization is formulated to penalize the mean squared difference between the prescribed and computed dose, other functions can be formulated with the methods of the said invention, for example using dose volume constraints for the structures of interest.

### 8.4 Modes of Carrying Out the Invention

### 8.4.1 Method of Radiation Delivery for Dynamic Trajectory Radiation Treatment (DTRT) Using Non-Colliding Dynamic Arc Trajectories

Accurate and efficient partial breast radiation requires careful planning of radiation treatment; this involves defining the location and the volume of the target tissue that should be treated. In the art, this volume is also routinely referred to as planning target volume (PTV). The delivery of high radiation doses to the target tissue, while minimizing exposure of surrounding healthy tissues to radiation, requires careful treatment planning that is focused on delivering the target radiation dose with the highest precision and accuracy possible. The availability of radiation therapy approaches that integrate three-dimensional imaging technology has facilitated to better define the boundaries of the target tissue that should be treated.

According to one aspect, the described invention provides a method of radiation delivery for dynamic trajectory radiation treatment (DTRT) based on non-colliding dynamic arc trajectories, comprising defining couch trajectory, gantry angles and multi-leaf collimator rotation and optimizing multi-leaf collimator beam apertures and delivered monitor units (MU) to achieve planning treatment volume prescription, to maximize dose conformality and to minimize the exposure of critical organs to harmful radiation.

According to one embodiment, a dynamic trajectory radiation treatment plan is defined as a set of control points: (G(MU),T(MU),C(MU),MLC(MU)). These control points prescribe the gantry angle, couch position, collimator rotation, and multi-leaf collimator leaf positions, respectively, in terms of the cumulative delivered monitor units. The delivery trajectory, (G(MU),T(MU),C(MU)), is determined that accomplishes the DTRT delivery goal, for example, by avoiding delivery angles that pass through especially sensitive organs, or spreading the dose over greater number of angles.

Couch positions and gantry angles are chosen so that throughout the treatment (a) the planned target volume is within the multi-leaf collimator (MLC) aperture field of view and (b) no collisions occur between the gantry and either the subject or the couch. The choice of collimator rotation can affect (a) and must, therefore, be selected appropriately. Moreover, because the couch and gantry can be moved independently of one another, the space of possible couch and gantry position combinations is quite large. It is therefore necessary to restrict this space when considering potential couch and gantry trajectories during DTRT treatment. To simplify the process, therapy trajectories are chosen for specific treatment sites and diseases of interest—in contrast to subject-specific trajectories.

### 8.4.2 Method of Partial Breast Irradiation Using Non-colliding Dynamic Arc Trajectories

According to another aspect, the described invention provides a method of partial breast irradiation using non-colliding dynamic arc trajectories and thus achieving an improved radiation dose distribution in subjects who have been diagnosed with early stage breast cancer and require breast radiation treatment.

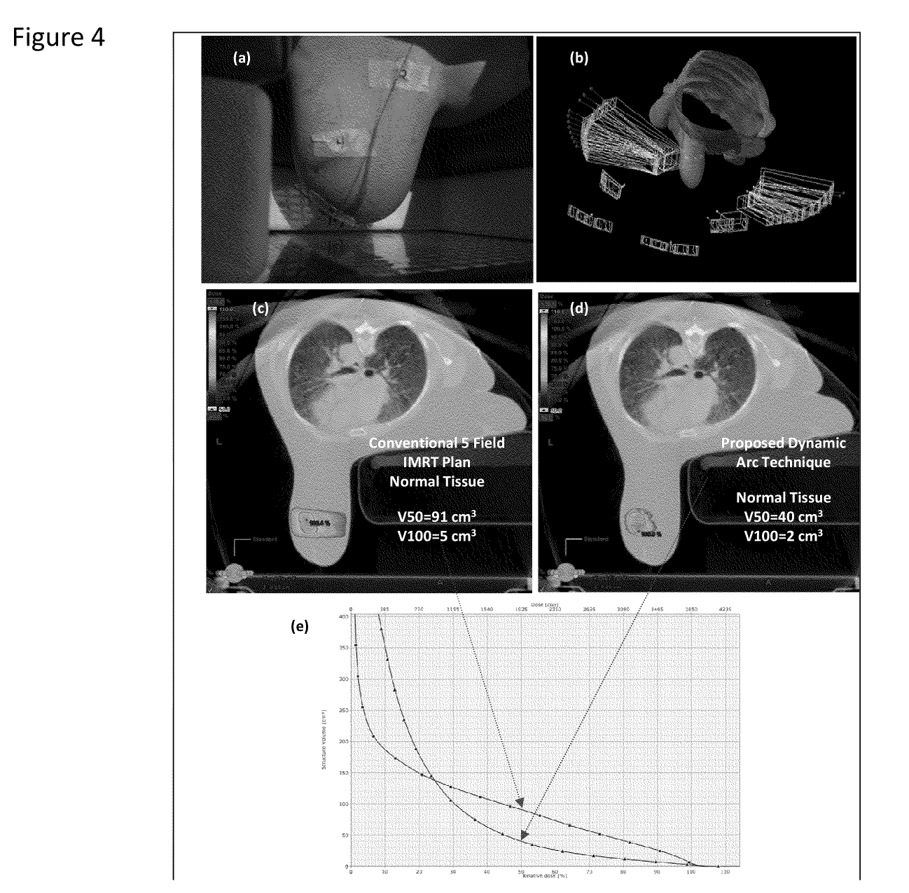

According to one embodiment, the present invention provides a method of partial breast irradiation which dynamically synchronizes motion of the gantry, multi-leaf collimator and couch to enable the creation of multiple horizontal, non-colliding arcs around the ipsilateral breast. Such horizontal (coronal) arc-like paths allow conformal irradiation of the diseased breast tissue alone without affecting surrounding healthy tissues and while sparing normal tissue beyond the chest wall. In one embodiment of the invention, the recipient of the partial breast irradiation is in a prone position, meaning that the breast(s) receiving radiation are pending downwards, away from neighboring tissues and organs. Using pending breast radiation beams, a conformal irradiation of diseased breast tissue such as a post-lumpectomy cavity is achieved with a highly conformal radiation dose distribution through wide angular irradiation (as shown in FIG. 4b).

Automated synchronization of gantry, couch and multi-leaf collimator is instrumental in producing non-colliding trajectories.

Embodiments of the present invention can be utilized with currently available structures of common medical linear accelerators (such as those marketed by Varian, Siemens, Elekta, etc.), if automated couch motion during treatment is enabled during treatment.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### 9. EXAMPLES

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

### Example 1

### Dynamic Accelerated Partial Breast Arc Irradiation via Synchronized Trajectories of Couch and Gantry Using Systems That Allow for Automated Movement of the Radiation Couch During Radiation Delivery

Accelerated partial breast irradiation (ABPI) is actively being evaluated as an alternative to whole breast irradiation particularly for women that were diagnosed with early stage breast cancer, see clinical trials such as the Radiation Therapy Oncology Group 0413 (RTOG 0413)/National Surgical Adjuvant Breast and Bowel Project (NSABP) B-39 Phase III clinical trial with a target subject sample size of 4,300 (Norris & Julian, 2008).

Early stage breast cancer is defined as stage II or less from a total of four stages or on the basis of the lack of lymph node, metastasis and clinical lesion size of 2 cm or less. With the availability of continuously improving breast cancer screening devices and methods, e.g. by mammography, early stage diagnosis of breast cancer is on the rise. Women who are diagnosed with early-stage breast cancer can chose between breast-conserving surgery (lumpectomy) plus post-operative adjuvant radiation treatment or full removal of the affected breast (mastectomy). In general, breast-conserving surgery followed by postoperative whole breast external beam radiation treatment over a period of weeks or months is nowadays the standard of care for subjects who are diagnosed with early breast cancer (Vaidya et al., 2010), unless genetic disposition or other circumstances call for a mastectomy.

Many, if not most cases of ipsilateral (same side) breast tumor recurrences following lumpectomy and whole-breast external beam radiation treatment occur close to the lumpectomy cavity which was the tumor center (Vaidya et al., 2010). This has called the necessity of whole breast irradiation into question. Accelerated partial breast irradiation (APBI) of the lumpectomy cavity is an approach that exposes only the lumpectomy bed plus a 1-2 cm tissue margin to radiation instead of the whole breast. In comparison to whole breast irradiation, in APBI daily fraction doses are increased.

There are invasive and non-invasive options of accelerated partial breast irradiation available. Brachytherapy is an invasive treatment modality which utilizes radioactive sources that are deposited into the affected breast tissue to expose the lumpectomy site to radiation. Intensity Modulated Radiation Therapy (IMRT) and 3D-Conformal Radiotherapy (3D-CRT) are non-invasive options for accelerated partial breast irradiation. However, with existing IMRT and 3D-CRT APBI approaches, unacceptably high rates of moderate-to-severe late normal tissue toxicities (˜20%) correlated to several dose-volume parameters have been reported (Chen et al., 2010; Hepel et al., 2009; Jagsi et al., 2010; Prosnitz et al., 2009). Specifically, the retrospective study of Jagsi et al. has shown that the occurrence of unacceptable cosmesis is correlated with the volume of normal breast tissue receiving 50% and 100% of the prescription dose (V50=19.25 Gy, V100=38.50 Gy) (Jagsi et al., 2010). Such toxicity can be attributed to the fact that current techniques, deploy isocentric tangential field arrangements that spare healthy organs, such as lungs and heart, but non-conformally irradiate large volumes of breast to moderate and high doses.

Angular and Geometrical Clearance Restrictions With Respect to Conformal Irradiation.

External beam accelerated partial breast irradiation (APBI) techniques are limited in achievable dose conformality because of angular and geometrical clearance restrictions of isocentric breast setups) Clinically, high rates of unacceptable cosmesis (10-20%) have been reported with such APBI techniques and attributed to normal tissue volume receiving 100% and 50% of the prescription dose of 38.5 Gy (10 fraction regiment, delivered twice daily) (Jagsi et al., 2010).

Utilizing a system that allows for automated movement of the radiation couch during radiation delivery, the general technique consists of the following.
A) Using information about the geometrical properties of the radiation delivery system, size of subject and immobilization apparatus (which can be derived from a simulation CT scan, for example), and user defined tolerances, the trajectories for couch rotation and translation, and corresponding gantry rotation, that avoid collision, are calculated. B) The trajectories comprise valid control points which are fed into a treatment planning algorithm which in turn determines the parameters for treatment delivery, such as MLC apertures and dose rate as function of couch and gantry location along the trajectory. The treatment planning algorithm can be of different types; it can, for example, be a 3D-conformal algorithm in which the multi-leaf collimator surrounds a BEV projection of the target, plus some user-defined margin, as shown in FIGS. 4a and 4b, and in general inverse planning presented in this invention, or algorithms existing in the art, fluence maps to be delivered by the MLC are determined. C) The optimized path, MLC apertures, gantry positions, and associated dose rates are programmed into the radiation delivery system for automated coordinated delivery.

The trajectory optimization for prone APBI dynamic delivery proceeds in 2-steps as outlined in Table 1, supra. First, for monotonous couch angle increments a set of horizontal beams is generated such that the target projection remains within the limits of the MLC, while collisions between the patient and the gantry are avoided. These horizontal beams are described by a set of control points, which capture couch translational parameters (LONG, LAT) as a function of the couch rotation (ROT). Subsequently, the beam weights and/or the intensities are optimized by inverse planning. For dynamic treatment delivery, the parameters of the optimized beams are mapped as functions of commutative beam-on time. A key component of the planning procedure is an algorithm, which we implemented within the PLUNC research TPS to design the couch trajectory (LONG (ROT), LAT (ROT)). The underlying principle is to use the MLC to track the target in the Beam Eye View (BEV) as the couch rotates and adjust the trajectory (LONG (ROT), LAT (ROT)) only when it is necessary to bring the target back within the MLC limits in the BEV. To test for collisions, an anatomical structure is introduced which encompasses treatment couch and patient anatomy. A 4D CAD model can be utilized to visualize or alternatively optimize the path, as demonstrated in the FIG. 3.

Relative to the current 5 field IMRT protocol shown in FIG. 4(c), the proposed dynamic arc method results in significantly smaller volume of the normal breast tissue receiving a higher dose. Specifically, the volume of normal tissue receiving 50% of the prescription (V50) was reduced by 44%, and measured to be a value of 91 cm³ to a value of 40 cm³ for the conventional 5 field IMRT plan, and the propose dynamic arc plan, respectively. Similarly, V100 was measured to be lower for the dynamic arc plan at a value of 2 cm³ versus the IMRT plan at 5 cm³. The above results are particularly clinically relevant, since as reported by Jagsi et al., 2010, there is a strong correlation of late side effects and the treatment plan V50 and V100. Specifically, V50 was lower in subjects with acceptable cosmesis, at 34.6%, than in those with unacceptable cosmesis, at 46.1%. Analogously, the mean V100 was also lower in subjects with acceptable side effects at cosmesis 15.5%, versus those with unacceptable side effects, at 23.0%.

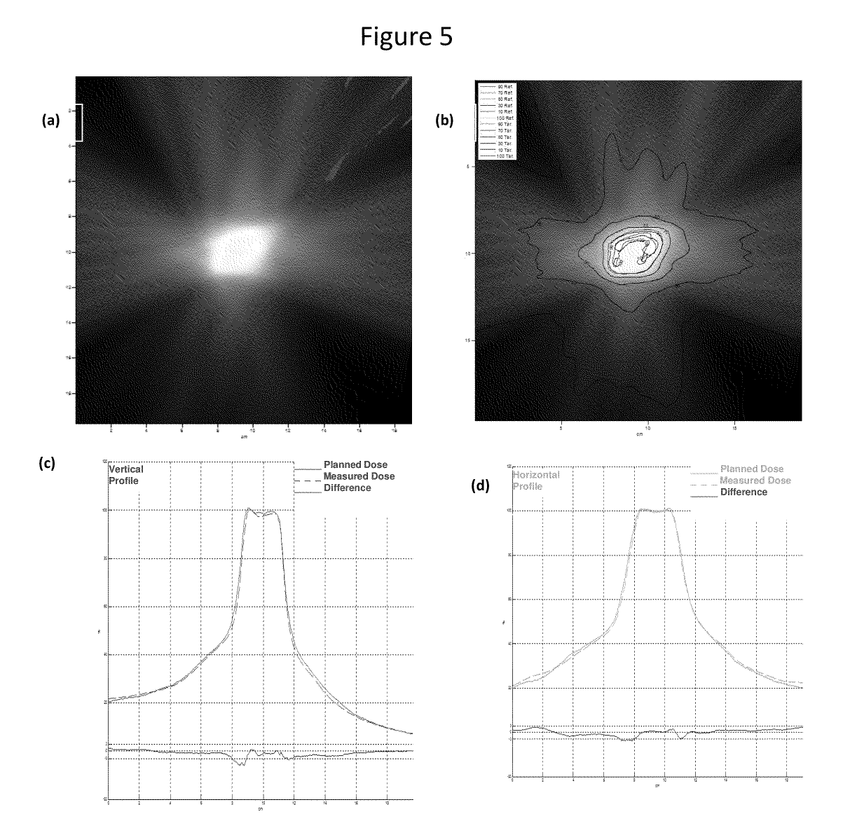

The DTRT technique for the said prone breast case was fully implemented on an existing LINAC. FIG. 4 shows the treatment planning results relative to conventional methods and demonstrates the benefit of reduction of normal tissue dose and associated dose parameters correlated to patient toxicity. As shown in FIG. 5, the calculated and delivered doses on the pinpoint chamber were within 3%, with an average deviation of 2.4%. EDR2 film measurements, placed 1 cm anterior and posterior to the target resulted in 90-100% of the points in the measured two-dimensional dose maps passing the 3%/3 mm Gamma criterion for the different measurements. Relative to the conventional 5 field IMRT protocol, in the studied cases, the proposed dynamic arc method resulted in up to 44% lower volume of normal tissue receiving 50% of the prescription (V50%), and 40% lower volume of normal tissue receiving 100% of the prescription (V100%).

The results of this study demonstrate that the proposed method of synchronous treatment trajectories of the couch and gantry is a feasible delivery method that can significantly improve APBI dose conformality, while reducing the exposure of normal, healthy tissue to radiation. In view of the recent data showing that the occurrence of unacceptable cosmesis is correlated with the volume of normal breast tissue receiving 50% and 100% of the prescription dose, this method may allow for more clinically favorable APBI treatments with considerably reduced toxicity.

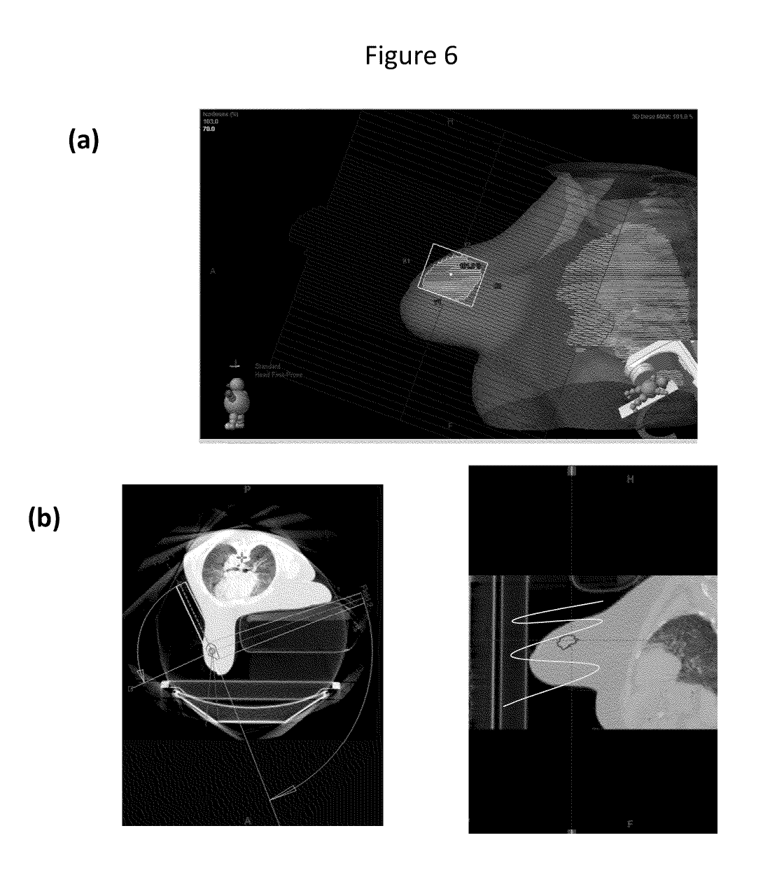

In alternative embodiments, the gantry can move in a coordinated manner through partial arcs while the couch is translating and rotating, as depicted by the yellow trajectories in FIG. 6b. The couch rotation and translation as a function of time can include pauses in movement during the trajectory (i.e., zero translation and/or rotation); this can occur, for example, when the couch does not translate, but rotates, and the MLC tracks the target, while the target is in the range of the field of view of the MLC.

### Example 2

### Tomographic Imaging via Dynamic Rotation of the Couch

Conventional computed tomographic (CT) imaging consists of a source, such as an x-ray tube, and a detector, rotating in the transverse plane of the subject to acquire projections at different source-to-subject angles, from which cross-sectional images of the 3D object are then reconstructed using mathematical algorithms Examples include Cone-Beam CT systems which consist of an on board imager attached to a LINAC that rotate about the stationary subject to produce cross-sectional images in the anatomical transverse plane. For imaging of the breast, such imaging would require the radiation from imaging source passing through all organs in the vicinity of the breast, such as lungs, heart, contra-lateral breast, etc., and delivering harmful radiation doses to these tissues.

Rather than moving the source about the subject in such a manner, in an aspect of the invention it is proposed that projections are acquired by having the source stationary, but inducing angular motion between the source and the imaging subject, by rotating the couch, and hence the imaging subject as shown in FIG. 2; while the imaged subject is being rotated, a detector at the opposite side of the source, detects the images, i.e., projections. The projections are then reconstructed using algorithms in the art for reconstruction in tomography or tomosynthesis. This would then produce cross-sectional images of the imaging subject in the anatomical coronal plane instead of the conventional transverse images.

In one embodiment of the present invention, a subject lays in a prone setup as shown in FIGS. 2a and 2b, with ipsilateral breast hanging below the trunk of the body. The imaging source is positioned to primarily irradiate the breast (see FIG. 2b). Keeping the source stationary, the treatment couch is rotated and translated, and images, i.e. projections, are acquired of the breast at different source-to-subject angles. This allows for a novel method to do ‘horizontal CT’ of the breast only, by which one avoids irradiating other tissues during the imaging procedure.

Variations of the invention include embodiments where multiple sets of projections (images) are acquired as described with the source being fixed at a different angle, the composite of which is reconstructed using tomographic algorithms As a limited number of projections (images) may be acquired, the reconstruction algorithm may be one of the various iterative regularized tomographic reconstruction algorithms that are better capable of limited angle tomographic reconstruction such as those by the inventors (Fahimian et al., 2010).

### Example 3

### Creation of Complex Non-Coplanar Curving Arc-Like Irradiation Paths via Dynamic Rotation of the Couch

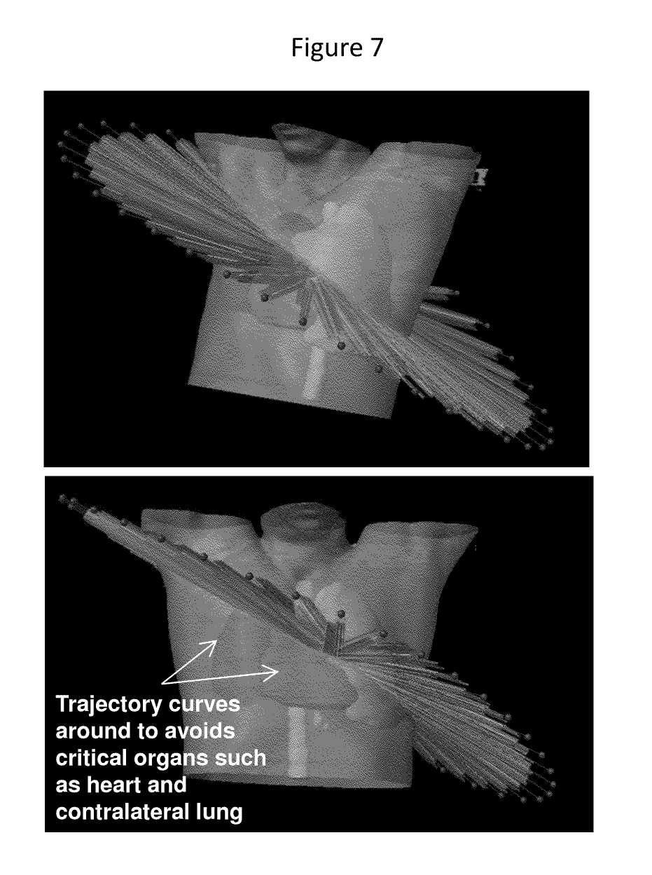

Another embodiment of DTRT is reflected in FIG. 7 which shows how DTRT can be utilized to produce complex non-coplanar curving arcs to minimize the exposure of healthy organs to harmful, unnecessary radiation and to increase degrees freedom in beam delivery through non-conventional beam angulation. In FIG. 7a, left lung treatment using a DTRT modulated arc technique is illustrated, using dynamic motion of the couch during gantry rotation, in which a trajectory is derived that minimizes beams impinging on the heart and contralateral lung. Other embodiments include the placement of slanted non-axial arcs through a combination of couch and gantry motions.

### Example 4

### Radiation Treatment of Large Anatomical Regions of the Body

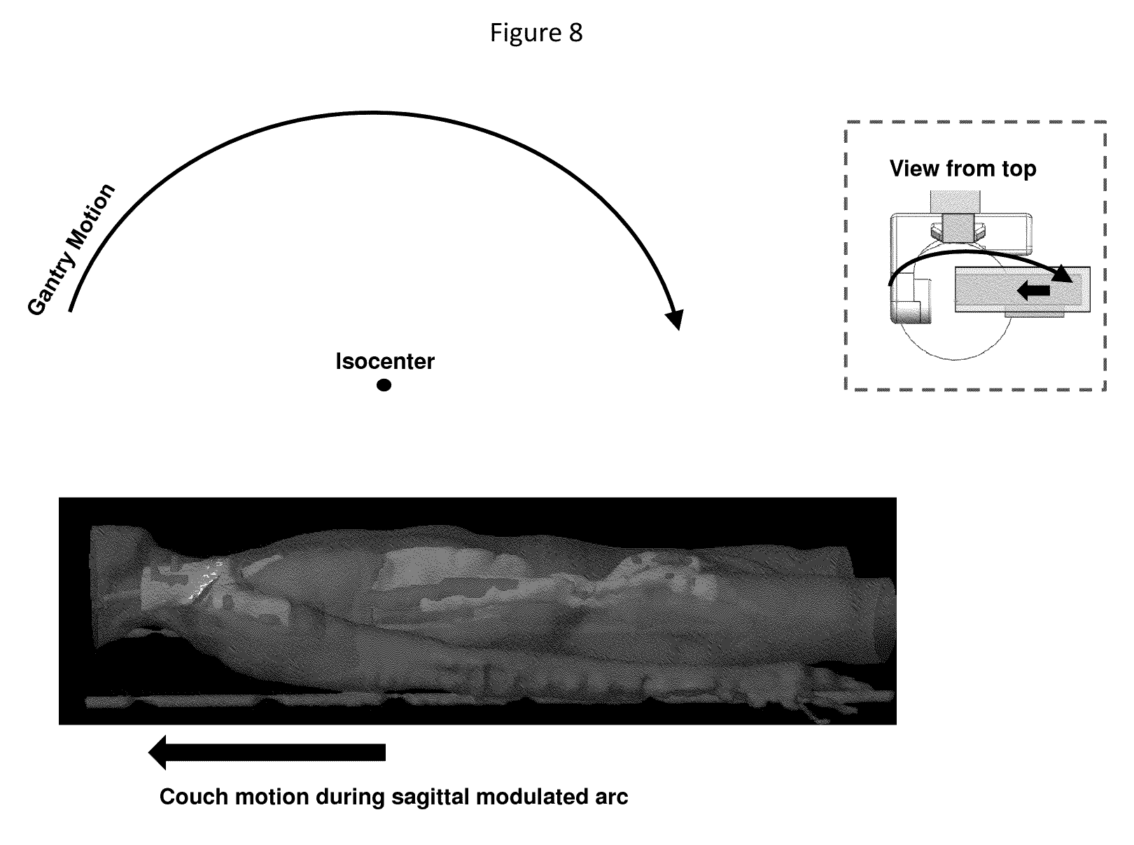

Another embodiment includes treatments in which large and extended portions of the body require radiation treatment. Under conventional arrangements, large areas of the body cannot be fitted under the beam, and special techniques such as multiple isocenters, or static types of treatments consisting of gantry pointing at a standing or sitting subject at extended distance are incorporated. Such treatments include total body irradiation (TBI) used for immune system suppression prior to bone marrow transplantation, or total lymphoid irradiation (TLI). Using a preferred embodiment of DTRT, large anatomical portions of the subject or the whole subject can be irradiated on the couch using concurrent motion of the couch and rotation of the gantry in a manner that utilizes the power of inverse planning and intensity modulated delivery. As shown in FIG. 8, the subject is positioned on a couch that is rotated either to 90 or 270 degree (IEC standard) and extended away from the source (extended Source to Skin Distance (SSD) setup). Then starting with the gantry tilted and a couch position such that the farthest anatomical region to be covered is in the beams eye view of the MLCs, the gantry is rotated while the couch is translated to the opposite extent. This allows full coverage of the large anatomical region in a single sagittal modulated partial arc. The process can be repeat analogously from the posterior-anterior direction by extending the couch upwards and delivering an arc beneath the couch (most modern therapy couches extend beyond their base, as shown in FIG. 1, allowing for the gantry to rotate below the couch).

Various types of radiation sources, e.g., photons of varying energy, electrons of varying energy, protons, neutrons, heavy ions, etc., and radiation delivery systems are contemplated to be utilized in the described embodiments. In addition, various other apparatus for immobilization and prone placement of subject can be utilized. In general, dynamic change in dose rate, as function of couch and gantry position is allowed, as in the case of RapidArc/VMAT. A variation of technique includes a supine subject setup, whereby through both translation and rotation of the couch non-isocentric arcs are delivered. Couch translation implies movement in all directions, including the vertical.

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Chen P Y et al. (2010). Four-year efficacy, cosmesis, and toxicity using three-dimensional conformal external beam radiation therapy to deliver accelerated partial breast irradiation. Int J Radiat Oncol Biol Phys 76:991-997.
Fahimian B P et al. (2010). Low-dose x-ray phase-contrast and absorption CT using equally sloped tomography. Phys Med Biol 55:5383-5400.
Hepel J T et al. (2009). Toxicity of three-dimensional conformal radiotherapy for accelerated partial breast irradiation. Int J Radiat Oncol Biol Phys 75:1290-1296.
Jagsi R et al. (2010). Unacceptable cosmesis in a protocol investigating intensity-modulated radiotherapy with active breathing control for accelerated partial-breast irradiation. Int J Radiat Oncol Biol Phys 76:71-78.
Njeh C F et al. (2010). Accelerated Partial Breast Irradiation (APBI): A review of available techniques. Radiat Oncol 5:90.
Norris & Julian (2008). Update on the NSABP B-39/RTOG 0413 Clinical Trial. Oncology Issues: 20-21.
Prosnitz L R et al. (2009). Accelerated partial breast irradiation: caution and concern from an ASTRO task force. Int J Radiat Oncol Biol Phys 74:981-984.
Vaidya J S et al. (2010). Targeted intraoperative radiotherapy versus whole breast radiotherapy for breast cancer (TARGIT-A trial): an international, prospective, randomized, non-inferiority phase 3 trial. The Lancet 376:91-102.

What is claimed is:

1. A method of delivering dose conformal radiation treatment to a subject suffering from a disease susceptible to radiation treatment, the method comprising
a) radiation treatment planning for delivering dose conformal radiation treatment comprising (i) defining radiation couch trajectory, gantry angles and multi-leaf collimator rotation; (ii) optimizing multi-leaf collimator beam apertures; (iii) optimizing delivered monitor units (MU) for forming non-colliding dynamic arc trajectories; and
b) delivering radiation treatment to diseased target tissue of said subject in accordance to radiation treatment planning.

2. The method according to claim 1, wherein the disease is a solid cancer.

3. The method according to claim 2, wherein the solid cancer is breast cancer.

4. The method according to claim 3, wherein radiation treatment is delivered in a partial breast irradiation setup.

5. The method according to claim 4, wherein the subject is in a prone position.

6. The method according to claim 1, wherein trajectory optimization algorithms and computer visualization techniques incorporating collisions detection of patient and delivery modules are utilized to determine optimized trajectories.

7. A method of radiation treatment planning for delivering dose conformal radiation treatment to a subject suffering from a disease susceptible to radiation treatment, the method comprising (i) defining radiation couch trajectory, gantry angles and multi-leaf collimator rotation; (ii) optimizing multi-leaf collimator beam apertures; (iii) optimizing delivered monitor units (MU) for forming non-colliding dynamic arc trajectories.

8. The method according to claim 7, wherein trajectory optimization algorithms and computer visualization techniques incorporating collisions detection of patient and delivery modules are utilized to determine optimized trajectories.

9. The method according to claim 7, wherein the disease is a solid cancer.

10. The method according to claim 9, wherein the solid cancer is breast cancer.

11. The method according to claim 10, wherein radiation treatment is delivered in a partial breast irradiation setup.

12. The method according to claim 11, wherein the subject is in a prone position.

13. A method of delivering dose conformal, partial breast radiation treatment to a subject suffering from breast cancer, the method comprising
a) radiation treatment planning comprising (i) defining radiation couch trajectory, gantry angles and multi-leaf collimator rotation; (ii) optimizing multi-leaf collimator beam apertures; (iii) optimizing delivered monitor units (MU) for forming a series of non-colliding dynamic complex coronal arc-like trajectories; and
b) delivering radiation treatment to diseased target tissue of said subject in accordance to radiation treatment planning.

14. The method according to claim 13, wherein the subject is in a prone position.

15. A method for imaging a region of interest in a subject comprising inducing relative motion between a radiation source and said subject via rotation and movement of a device for supporting said subject for acquiring images at different source-to-subject angles.

16. The method of claim 16, wherein said images are used for tomographic or tomosynthesis reconstruction to produce cross-sectional images of said region of interest in the subject.

17. A method of delivering dose conformal radiation treatment to a subject suffering from a disease susceptible to radiation treatment and requiring radiation treatment for large and extended body portions, the method comprising
producing non-coplanar curving arcs through motion of couch during gantry rotation;
and increasing degrees of freedom in beam delivery through non-conventional beam angulation as to minimize exposure of healthy tissues and organs to harmful and unnecessary radiation.

18. A system for imaging a subject comprising a radiation source and a device for supporting said subject suitable for acquiring images at different source-to-subject angles.
